(19) United States
(12) Patent Application Publication
(10) Pub. No.: US 2011/0217207 A1
(43) Pub. Date: Sep. 8, 2011

(54) ANALYSIS TOOL, IDENTIFICATION APPARATUS, AND ANALYSIS APPARATUS

(75) Inventors: Koji Fujimoto, Kyoto-shi (JP); Noriaki Furusato, Kyoto-shi (JP); Hideko Kosaka, Kyoto-shi (JP)

(73) Assignee: ARKRAY, INC., Kyoto-shi (JP)

(21) Appl. No.: 13/039,235

(22) Filed: Mar. 2, 2011

(30) Foreign Application Priority Data
Mar. 2, 2010 (JP) 2010-045879

(51) Int. Cl. G01N 21/64; G01N 33/00; G01N 21/00

(52) U.S. Cl. 422/82.08; 422/68.1; 422/82.05

(57) ABSTRACT

A technique is provided, which makes it possible to easily judge whether or not an analysis tool to be used for an analysis process for a sample is authentic and which makes it possible to secure the reliability of an analysis result thereby. An identification apparatus for identifying an analysis tool having a reagent portion to be used for an analysis process for a sample is provided, wherein the analysis tool further includes an identifying marker portion which is formed by using an invisible substance, the identification apparatus comprising a light emitter which emits a light toward the identifying marker portion, a light receiver which receives a light emitted by the invisible substance for forming the identifying marker portion exposed with the emitted light emitted by the light emitter, and an identification unit which identifies the analysis tool on the basis of a light-receiving result obtained by the light receiver.

### CROSS-REFERENCE TO RELATED APPLICATION

The present application claims priority to Japanese patent applications No. JP2010-045879, filed on Mar. 2, 2010 the disclosure of which is incorporated herein by reference in its entirety.

### FIELD

The present embodiments relates to an analysis tool, an identification apparatus, and an analysis apparatus.

### DESCRIPTION OF THE RELATED ART

Conventionally, an analysis tool (analysis kit, analysis instrument) such as a test paper, a test strip or the like which is used to analyze a sample (specimen) such as urine, blood or the like and an analysis apparatus which analyzes the sample by using the analysis instrument are known (see, for example, Japanese Patent Application Laid-open Nos. 2000-321270, 2009-229232, and 7-35744).

An analysis process for analyzing the sample is performed by the analysis apparatus, for example, such that the color development, which is brought about when a reagent portion (reagent pad or the like) containing a reagent to cause a reaction with the sample is impregnated with the sample, is optically measured and observed by using an optical technique.

In order to secure the reliability of the analysis result of the sample obtained by the analysis apparatus, it is premised that the analysis process is performed by using the analysis tool which is provided as the so-called regular product (genuine product) for which the quality is guaranteed by a manufacturer or the like. However, it is not easy for a user (operator) of the analysis apparatus to distinguish the regular product from any imitation or copy (fake, forgery, sham) which is produced to have an appearance similar to that of the regular product. For example, it is feared that an imitation analysis tool (hereinafter referred to as “forged analysis tool” as well) may be erroneously recognized as the regular product analysis tool (hereinafter referred to as “regular analysis tool” as well) and the forged analysis tool may be used for the analysis process. Therefore, it is feared that the reliability of the analysis result may be lowered, for example, such that the analysis error may be increased for the sample. As a result, it is feared that any examination mistake (inspection mistake) may be caused, for example, such that the examination may fail to notice a sickness (illness) or a disease of an examinee.

### SUMMARY OF THE INVENTION

The present embodiments provide a technique which makes it possible to easily judge whether or not an analysis tool to be used for an analysis process for a sample is authentic and which makes it possible to secure the reliability of an analysis result thereby.

In certain embodiments, the following means can be adopted in order to solve the problem as described above.

For example, the analysis tool according to the present embodiment resides in an analysis tool having a reagent portion to be used for an analysis process for a sample; the analysis tool comprising an identifying marker portion which is formed by using an invisible substance.

The reagent portion contains a reagent to be reacted with the sample. The numerical information, which includes, for example, the concentration in relation to any specified component contained in the sample, is calculated on the basis of a measurement result obtained by optically measuring the coloration state provided when the reagent portion is impregnated with the sample, for example, by using any optical technique.

According to the analysis tool concerning the present invention, the identifying marker portion is formed by using the invisible substance. The invisible substance is a substance through which the so-called visible light is transmitted. The invisible substance is, for example, an invisible ink (paint) which is recognized or grasped to be colorless under the visible light. Therefore, the identifying marker portion is not read by naked eye under the visible light. In other words, even if the analysis tool according to the present invention is faked or imitated, the identifying marker portion is never replicated. Therefore, when the identifying marker portion is unsuccessfully read by using, for example, any optical technique, it is meant thereby that the identifying marker portion, which should be present if the analysis instrument is a regular test strip, is absent. Therefore, it is possible to discriminate that the concerning test strip is a fake or an imitation.

In the analysis tool according to the present invention, the identifying marker portion may be formed by using a fluorescent substance which emits fluorescence by using an ultraviolet ray as an exciting light. In this arrangement, the identifying marker portion may be provided on a front surface or a back surface of a base member, and at least an area of the base member, in which the identifying marker portion is provided, may be formed of a material through which the ultraviolet ray is not transmitted and through which the fluorescence emitted by the fluorescent substance is transmitted.

In the analysis tool according to the present invention, the identifying marker portion is provided while being embedded in the base member, and a portion disposed on a side of one surface may be formed of a material through which the ultraviolet ray is transmitted and a portion disposed on a side of the other surface may be formed of a material through which the ultraviolet ray is not transmitted and through which the fluorescence emitted by the fluorescent substance is transmitted, with a boundary disposed at an embedding position of the identifying marker portion in a thickness direction of the base member in at least an area of the base member in which the identifying marker portion is embedded.

Identification information about the analysis tool may be recorded by using the invisible substance on at least a part of the identifying marker portion. The identification information is information provided to identify the analysis tool to be used for an analysis apparatus. The identification information may include product specification information about the analysis tool and/or the identification information may include analysis-related information to be used when the analysis process is performed for the sample. The product specification information can be exemplified, for example, by the production lot and the manufacturer's serial number of the analysis tool. The analysis-related information can be exemplified, for example, by the type of the sample to be used as the analysis objective, the type of the measuring component (specified component), and the identification mark corresponding to the so-called calibration curve data or the working curve data stored, for example, in a memory of a computer of, for example, the analysis apparatus for performing the analysis process for the sample or the identification apparatus for performing the identification of the analysis tool.

In another aspect, the present invention can be comprehended as an identification apparatus for identifying the analysis tool. That is, the identification apparatus according to the present invention resides in an identification apparatus for identifying an analysis tool having a reagent portion to be used for an analysis process for a sample; wherein the analysis tool further includes an identifying marker portion which is formed by using an invisible substance; the identification apparatus comprising a light emitter which emits a light toward the identifying marker portion; a light receiver which receives a light emitted by the invisible substance for forming the identifying marker portion exposed with the emitted light emitted by the light emitter; and an identification unit which identifies the analysis tool on the basis of a light-receiving result obtained by the light receiver. In this arrangement, the identification unit can identify the analysis tool by judging whether or not the analysis tool is authentic, on the basis of the light-receiving result obtained by the light receiver.

According to the identification apparatus constructed as described above, in the case of the regular (formal or proper) analysis tool, the light receiver can receive the light (including the reflected light as well) emitted by the invisible substance of the identifying marker portion exposed with the emitted light emitted by the light emitter. On the other hand, the identifying marker portion is absent from the beginning in the case of any forged analysis tool. Therefore, the light receiver never receives the light emitted by the invisible substance. Therefore, it is possible to easily and highly accurately identify whether or not the analysis tool is the regular product, depending on the light-receiving result obtained by the light receiver, for example, the presence or absence of the light received by the light receiver. Accordingly, it is possible for a user to avoid any unintentional use of the forged analysis tool in the analysis process, which would be otherwise caused such that the forged analysis tool is erroneously recognized as the regular analysis tool. Thus, it is possible to suppress the occurrence of any inconvenience which would be otherwise caused, for example, such that the analysis error is increased for the sample by using the analysis tool, and it is possible to avoid the deterioration of the reliability of the analysis result. Further, it is possible to more reliably avoid the occurrence of any examination mistake, which would be otherwise caused, for example, such that the sickness (illness) or the disease of an examinee or a subject is not found and noticed.

In relation to the analysis tool to be identified by the identification apparatus according to the present invention, the identifying marker portion may be formed of a fluorescent substance which emits fluorescence by using an ultraviolet ray as an exciting light, the light emitter may emit the ultraviolet ray, and the light receiver may receive the fluorescence emitted by the fluorescent substance for forming the identifying marker portion.

In relation to the analysis tool, when the identifying marker portion is provided on a front surface of a base member of the analysis tool, and at least an area of the base member, in which the identifying marker portion is provided, is formed of a material through which the ultraviolet ray is not transmitted and through which the fluorescence emitted by the fluorescent substance is transmitted, then the light emitter of the identification apparatus may emit the ultraviolet ray from a side of the front surface of the base member, and the light receiver may receive the fluorescence transmitted through the base member on a side of a back surface of the base member.

In relation to the analysis tool, when the identifying marker portion is provided while being embedded in the base member of the analysis tool, a portion disposed on a side of one surface is formed of a material through which the ultraviolet ray is transmitted, and a portion disposed on a side of the other surface is formed of a material through which the ultraviolet ray is not transmitted and through which the fluorescence emitted by the fluorescent substance is transmitted, with a boundary disposed at an embedding position of the identifying marker portion in a thickness direction of the base member in at least an area of the base member in which the identifying marker portion is embedded, then the light emitter of the identification apparatus may emit the ultraviolet ray from the side of the one surface of the base member, and the light receiver may receive the fluorescence transmitted through the base member on the side of the other surface of the base member.

In still another aspect, the present invention may reside in an analysis apparatus or an analysis system comprising any identification apparatus as described above, wherein the analysis process is performed for the sample by using the analysis tool identified by the identification apparatus. Even when the analysis apparatus or the analysis system as described above is used, it is also possible to obtain the effects which are the same as or equivalent to those of the features referred to in relation to the identification apparatus according to the present invention.

In still another aspect, the present invention can be comprehended as an identification method for identifying any analysis tool as described above. That is, the identification method according to the present invention resides in an identification method for identifying an analysis tool having a reagent portion to be used for an analysis process for a sample; wherein the analysis tool further includes an identifying marker portion which is formed by using an invisible substance; the identification method comprising a light-emitting step of emitting a light toward the identifying marker portion; a light-receiving step of receiving a light emitted by the invisible substance for forming the identifying marker portion exposed with the emitted light in the light-emitting step; and an identifying step of identifying the analysis tool on the basis of a light-receiving result obtained in the light-receiving step. According to the identification method as described above, it is possible to obtain the effects which are the same as or equivalent to those of the features referred to in relation to the identification apparatus according to the present invention. In the identifying step, it is possible to identify the analysis tool by judging whether or not the analysis tool is authentic, on the basis of the light-receiving result obtained in the light-receiving step.

In relation to the analysis tool to which the identification method according to the present invention is applied, when the identifying marker portion is formed of a fluorescent substance which emits fluorescence by using an ultraviolet ray as an exciting light, the identification method according to the present invention may further comprise emitting the ultraviolet ray in the light-emitting step, and receiving the fluorescence emitted by the fluorescent substance for forming the identifying marker portion in the light-receiving step.

In this procedure, when the identifying marker portion is provided on a front surface of a base member of the analysis tool, and at least an area of the base member, in which the identifying marker portion is provided, is formed of a material through which the ultraviolet ray is not transmitted and through which the fluorescence emitted by the fluorescent substance is transmitted, then the ultraviolet ray may be emitted from a side of the front surface of the base member in the light-emitting step, and the fluorescence transmitted through the base member may be received on a side of a back surface of the base member in the light-receiving step. Further, when the identifying marker portion is provided while being embedded in the base member of the analysis tool, a portion disposed on a side of one surface is formed of a material through which the ultraviolet ray is transmitted, and a portion disposed on a side of the other surface is formed of a material through which the ultraviolet ray is not transmitted and through which the fluorescence emitted by the fluorescent substance is transmitted, with a boundary disposed at an embedding position of the identifying marker portion in a thickness direction of the base member in at least an area of the base member in which the identifying marker portion is embedded, then the ultraviolet ray may be emitted from the side of the one surface of the base member in the light-emitting step, and the fluorescence transmitted through the base member may be received on the side of the other surface of the base member in the light-receiving step.

In still another aspect, the present invention can be comprehended as an identification program to be executed by a computer in order to identify any analysis tool as described above. That is, the identification program according to the present invention resides in an identification program to be executed by a computer in order to identify an analysis tool having a reagent portion to be used for an analysis process for a sample; wherein the analysis tool further includes an identifying marker portion which is formed by using an invisible substance; the identification program allowing the computer to execute a light-emitting step of emitting a light toward the identifying marker portion; a light-receiving step of receiving a light emitted by the invisible substance for forming the identifying marker portion exposed with the emitted light in the light-emitting step; and an identifying step of identifying the analysis tool on the basis of a light-receiving result obtained in the light-receiving step. In the identifying step, it is possible to identify the analysis tool by judging whether or not the analysis tool is authentic, on the basis of the light-receiving result obtained in the light-receiving step. According to the identification program as described above, it is possible to obtain the effects which are the same as or equivalent to those of the features referred to in relation to the identification apparatus according to the present invention. In still another aspect, the present invention can be also comprehended as a recording medium including the identification program recorded thereon, the recording medium being readable by a computer.

According to still another aspect of the present invention, there is provided an analysis apparatus for performing an analysis process for a sample by using an analysis tool having a reagent portion; wherein the analysis tool has an identifying marker portion which is formed by using an invisible substance, the identifying marker portion having at least a part thereof on which analysis-related information to be used when the analysis process is performed for the sample is recorded; and the analysis apparatus comprising a light emitter which emits a light toward the identifying marker portion; a light receiver which receives a light emitted by the invisible substance for forming the identifying marker portion exposed with the emitted light emitted by the light emitter; an identification unit which identifies the analysis tool on the basis of a light-receiving result obtained by the light receiver; a storage unit which acquires the analysis-related information on the basis of the light-receiving result obtained by the light receiver and which stores the acquired analysis-related information; a measuring unit which measures the sample located on the reagent portion of the analysis tool; and a calculating unit which calculates numerical information in relation to a specified component contained in the sample on the basis of a measurement result of the sample measured by the measuring unit and the analysis-related information stored by the storage unit. The identification unit can identify the analysis tool by judging whether or not the analysis tool is authentic, on the basis of the light-receiving result obtained by the light receiver.

According to still another aspect of the present invention, there is provided an analysis system for performing an analysis process for a sample by using an analysis tool having a reagent portion; wherein the analysis tool has an identifying marker portion which is formed by using an invisible substance, the identifying marker portion having at least a part thereof on which analysis-related information to be used when the analysis process is performed for the sample is recorded; and the analysis system comprising an identification device including a light emitter which emits a light toward the identifying marker portion, a light receiver which receives a light emitted by the invisible substance for forming the identifying marker portion exposed with the emitted light emitted by the light emitter, and an identification unit which identifies the analysis tool on the basis of a light-receiving result obtained by the light receiver; a storage device which acquires the analysis-related information on the basis of the light-receiving result obtained by the light receiver and which stores the acquired analysis-related information; a measuring device which measures the sample located on the reagent portion of the analysis tool; and a calculating device which calculates numerical information in relation to a specified component contained in the sample on the basis of a measurement result of the sample measured by the measuring device and the analysis-related information stored by the storage device. The identification unit can identify the analysis tool by judging whether or not the analysis tool is authentic, on the basis of the light-receiving result obtained by the light receiver.

In still another aspect, the present invention can be comprehended as an analysis method for performing an analysis process for a sample by using any analysis tool as described above or an analysis program to be executed by a computer in order to perform the analysis process. That is, the analysis method according to the present invention resides in an analysis method for performing an analysis process for a sample by using an analysis tool having a reagent portion; wherein the analysis tool has an identifying marker portion which is formed by using an invisible substance, the identifying marker portion having at least a part thereof on which analysis-related information to be used when the analysis process is performed for the sample is recorded; and the analysis method comprising a light-emitting step of emitting a light toward the identifying marker portion; a light-receiving step of receiving a light emitted by the invisible substance for forming the identifying marker portion exposed with the emitted light in the light-emitting step; an identifying step of identifying the analysis tool on the basis of a light-receiving result obtained in the light-receiving step; a storing step of acquiring the analysis-related information on the basis of the light-receiving result obtained in the light-receiving step and storing the acquired analysis-related information; a measuring step of measuring the sample located on the reagent portion of the analysis tool; and a calculating step of calculating numerical information in relation to a specified component contained in the sample on the basis of a measurement result of the sample measured in the measuring step and the analysis-related information stored in the storing step.

According to still another aspect of the present invention, there is provided an analysis program to be executed by a computer for performing an analysis process for a sample by using an analysis tool having a reagent portion; wherein the analysis tool has an identifying marker portion which is formed by using an invisible substance, the identifying marker portion having at least a part thereof on which analysis-related information to be used when the analysis process is performed for the sample is recorded; and the identification program allowing the computer to execute a light-emitting step of emitting a light toward the identifying marker portion; a light-receiving step of receiving a light emitted by the invisible substance for forming the identifying marker portion exposed with the emitted light in the light-emitting step; an identifying step of identifying the analysis tool on the basis of a light-receiving result obtained in the light-receiving step; a storing step of acquiring the analysis-related information on the basis of the light-receiving result obtained in the light-receiving step and storing the acquired analysis-related information; a measuring step of measuring the sample located on the reagent portion of the analysis tool; and a calculating step of calculating numerical information in relation to a specified component contained in the sample on the basis of a measurement result of the sample measured in the measuring step and the analysis-related information stored in the storing step. It is possible to identify the analysis tool by judging whether or not the analysis tool is authentic, on the basis of the light-receiving result obtained in the light-receiving step, in the identifying step included in the analysis method or the analysis program as described above. In still another aspect, the present invention can be also comprehended as a recording medium including the analysis program as described above recorded thereon, the recording medium being readable by a computer.

In one embodiment, the analysis tool comprises a reagent portion configured to conduct an analysis of a sample, and an identifying marker portion comprising an invisible substance. In one embodiment, the identifying marker portion comprises a fluorescent substance configured to emit fluorescence in response to exposure to an ultraviolet ray. In one embodiment, the identifying marker portion is located on a front surface or a back surface of a base member, and at least an area of the base member, in which the identifying marker portion is provided, comprises a material configured to not transmit the ultraviolet ray and transmit the fluorescence emitted by the fluorescent substance. In one embodiment the identifying marker portion is embedded in a base member, and a first portion disposed on a side of one surface comprises a first material configured to transmit ultraviolet ray and a second portion disposed on a side of the other surface is formed of a second material configured to not transmit the ultraviolet ray and transmit the fluorescence emitted by the fluorescent substance, with a boundary embedded in the identifying marker portion in a thickness direction of the base member in at least an area of the base member. In one embodiment at least apart of the invisible substance is configured to record identification information about the analysis tool. In one embodiment, the identification information comprises at least any one of product specification information about the analysis tool and analysis-related information to be used when the analysis process is performed for the sample.

In another embodiment, the identification apparatus for identifying an analysis tool comprises a reagent portion to be used for an analysis process for a sample, an identifying marker portion comprising an invisible substance, a light emitter configured to emit a light toward the identifying marker portion, a light receiver configured to receive a light emitted by the invisible substance, wherein the light receiver is positioned on the identifying marker portion, and an identification unit configured to identify the analysis tool based on the light-receiving result obtained by the light receiver. In one embodiment the identifying marker portion comprises a fluorescent substance configured to emit fluorescence in response to excitation by an ultraviolet ray, the light emitter is configured to emit the ultraviolet ray, and the light receiver is configured to receive the fluorescence emitted by the fluorescent substance.

It is noted that the means for solving the problem according to the present invention can be combined with each other as far as possible. According to the present invention, it is possible to provide the technique which makes it possible to easily judge whether or not the analysis tool to be used for the analysis process for the sample is authentic and which makes it possible to secure the reliability of the analysis result thereby.

### BRIEF DESCRIPTION OF THE DRAWINGS

FIG. 1 shows a schematic perspective view illustrating an analysis test strip according to a first embodiment.

FIG. 2 shows a schematic perspective view illustrating an analysis apparatus according to the first embodiment.

FIG. 3 shows a structural block diagram illustrating an arrangement of the analysis apparatus according to the first embodiment.

FIG. 4 shows a part of the internal structure of the analysis apparatus according to the first embodiment.

FIG. 5 shows a structural block diagram illustrating a detailed arrangement of a test strip identification apparatus according to the first embodiment.

FIG. 6 illustrates the positional relationship between the test strip and respective devices of the test strip identification apparatus according to the first embodiment.

FIG. 7 shows a flow chart illustrating the contents of a process flow to be executed by a control computer of the test strip identification apparatus.

FIG. 8 shows an arrangement of a test strip according to a second modified embodiment, wherein FIG. 8A shows a front surface of a base member of the test strip, and FIG. 8B shows a sectional view taken along the longitudinal direction of the test strip.

FIG. 9 illustrates the positional relationship between the test strip and respective devices of a test strip identification apparatus according to the second modified embodiment.

FIG. 10 shows a schematic perspective view illustrating an analysis test strip according to a second embodiment.

FIG. 11 shows a functional block diagram illustrating an analysis apparatus according to the second embodiment.

FIG. 12 shows a flow chart illustrating the contents of a process flow to be executed by the analysis apparatus according to the second embodiment.

FIG. 13 illustrates the positional relationship between a test strip and respective devices of a test strip identification apparatus according to a third modified embodiment.

FIG. 14 illustrates the positional relationship between a test strip and respective devices of a test strip identification apparatus according to a fourth modified embodiment.

### DESCRIPTION OF THE PREFERRED EMBODIMENTS

Embodiments for carrying out the invention will be explained in detail below by way of example with reference to the drawings. In the embodiments of the present invention, the analysis tool according to the present invention, the identification apparatus for identifying the analysis tool, and the analysis apparatus provided with the identification apparatus will be explained by way of example. The explanation of the embodiments described below also illustrates respective embodiments in relation to the identification method for identifying the analysis tool (analysis kit, analysis instrument) according to the present invention, the identification program, the analysis method for performing the analysis process for a sample by using the analysis tool, the analysis system, the analysis program, and the recording medium which records the program thereon and which is readable by a computer. For example, the sizes, the materials, the shapes, and the relative arrangement thereof concerning the constitutive elements described in the embodiments of the present invention are not intended to limit the technical scope of the invention only thereto, unless otherwise specifically noted.

### First Embodiment

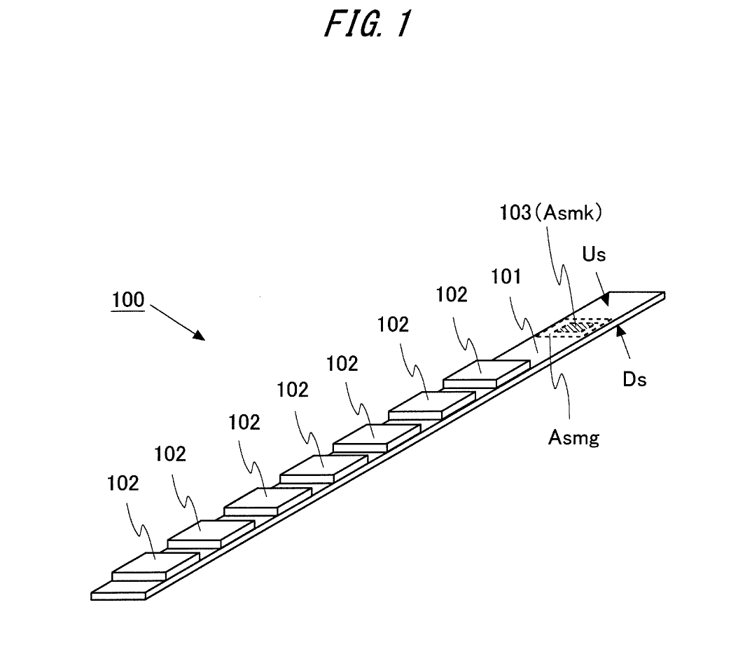

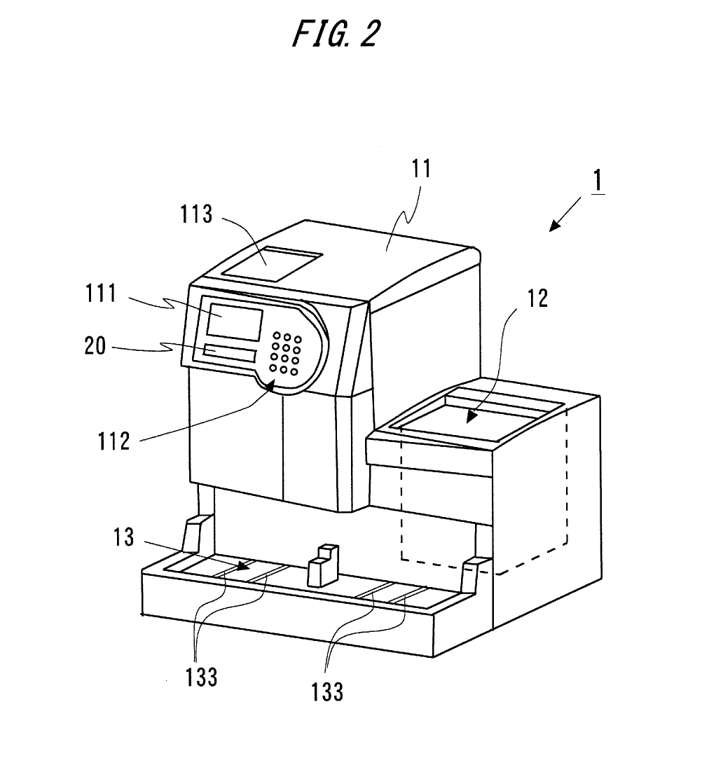

FIG. 1 shows a schematic perspective view illustrating an analysis test strip or test piece (hereinafter simply referred to as “test strip”) 100 provided as an analysis instrument or analysis tool according to a first embodiment. FIG. 2 shows a schematic perspective view illustrating an analysis apparatus 1 according to the first embodiment. The test strip 100 is an example of the analysis instrument to be used for the analysis process for a sample performed by the analysis apparatus 1 as shown in FIG. 2. The analysis apparatus 1 is an apparatus for performing the analysis process for the sample by using the test strip 100. In this embodiment, urine is adopted as the sample by way of example to explain the analysis apparatus 1 which performs the urine qualitative test by analyzing chemical components contained in the urine.

As shown in FIG. 1, the test strip 100 has a base member 101 which is thin plate-shaped and strip-shaped. A plurality of reagent pads (reagent portions) 102 are provided and aligned in the longitudinal direction of the surface of the base member 101 indicated by a symbol Us. The reagent pads 102 are impregnated or applied with reagents which cause the coloration by reacting with the urine components including, for example, glucose, protein, occult blood, and bilirubin. The seven reagent pads are conveniently provided on the base member 101 in FIG. 1. However, it is of course allowable to appropriately increase or decrease the number thereof. In this embodiment, the surface Us, on which the reagent pads 102 are formed, is conveniently designated as the “front surface” of the base member 101, and the surface, which is opposed to the front surface Us, is conveniently designated as the “back surface” Ds of the base member 101.

An identifying marker portion (hereinafter referred to as “identifying invisible marker portion”) 103, which is formed by using an invisible substance that cannot be recognized visually or that can be recognized visually extremely hardly, is provided on the front surface Us of the base member 101 (hereinafter abbreviated as “base member front surface” or “front surface of the base member” as well). In this case, the “marker” of the marker portion means, for example, the indicator, the index, or the mark. The invisible substance, which is used to form the identifying invisible marker portion 103, is the substance through which the so-called visible light is transmitted (i.e., visible light is neither absorbed nor scattered). The invisible substance is, for example, an invisible ink (paint, dye) which is grasped or recognized to be colorless under the visible light. In this embodiment, a fluorescent ink (corresponding to the fluorescent substance), which is excited by using the ultraviolet ray (ultraviolet light) as the exciting light to emit the fluorescence of the visible light, is used for the identifying invisible marker portion 103. The fluorescent ink may be an organic fluorescent dye or an inorganic fluorescent dye. Alternatively, the fluorescent ink may be a known single fluorescent dye or a mixture of two or more types of fluorescent dyes. The organic fluorescent dye may be exemplified, for example, by those based on stilbene, naphthalene, thiophene, and imidazolone; derivatives of heterocyclic 5-membered rings (those based on, for example, imidazole, triazole, and oxazole); and derivatives of heterocyclic 6-membered rings (those based on, for example, triazine, coumarin, and pyridine). The inorganic fluorescent dye may be exemplified, for example, by those based on zinc and rare earth.

However, it is also allowable that the light, which is emitted by the fluorescent ink (paint), is not the visible light, and there is no limitation to any special wavelength. It is also unnecessary that the exciting light should be the ultraviolet ray. The exciting light may be, for example, an infrared ray (infrared light). That is, it is possible to adopt various substances as the fluorescent ink for forming the identifying invisible marker portion 103 provided that the substance cannot be visually recognized by the naked eye under the visible light (including such embodiments that the substance can be visually recognized extremely hardly as well (this condition also holds in the following description)). The identifying invisible marker portion 103 may be formed by applying the fluorescent ink described above to the base member 101 or by adopting a method of printing formation or any other method.

It is now defined that the direction, which is parallel to the front surface Us and the back surface Ds of the base member 101, is the “in-plane direction (of the base member)”. Further, it is defined that the area (hatched area in FIG. 1), which is provided with (arranged with) the identifying invisible marker portion 103 in the in-plane direction of the base member 101, is the “marker portion arrangement area Asmk”. At least the marker portion arrangement area Asmk of the base member 101 of the test strip 100 is formed of a material or an ingredient (hereinafter referred to as “ultraviolet ray-intransmissive and fluorescence-transmissive type material”) through which the ultraviolet ray is not transmitted and through which the florescence emitted by the fluorescent ink for forming the identifying invisible marker portion 103 is transmitted. In this embodiment, the ultraviolet ray-intransmissive and fluorescence-transmissive type material is used for the marker portion arrangement area Asmk in the in-plane direction of the base member 101 and a predetermined margin area Asmg which surrounds the same. However, it is also allowable to use the ultraviolet ray-intransmissive and fluorescence-transmissive type material is used for the entire base member 101. A known ultraviolet blocking resin may be adopted as the ultraviolet ray-intransmissive and fluorescence-transmissive type material of the base member 101. In this embodiment, a PET film is adopted in consideration of the easy availability and the easily processable performance. However, there is no limitation thereto. Other than the PET film, for example, acrylic resins, which are represented, for example, by polystyrene and polymethylmethacrylate, can be also preferably adopted as the ultraviolet ray-intransmissive and fluorescence-transmissive type material.

The identifying invisible marker portion 103, which is provided on the base member 101 with the invisible fluorescent ink in relation to the test strip 100 constructed as described above, is utilized when the test strip 100 is identified by means of a test strip identification apparatus carried on the analysis apparatus 1. The identification of the test strip 100 referred to herein resides, for example, in that it is judged whether the test strip 100, which is used for the analysis process, is the regular test strip (hereinafter referred to, for example, as “regular test strip”, “regular product”, or “genuine product”) or any test strip (hereinafter referred to, for example, as “forged test strip”, “fake”, “imitation”, or “forgery or forged product”) produced by imitating the regular test strip. The regular test strip is the test strip as the genuine product for which the quality is guaranteed, for example, by a manufacturer.

The test strip identification apparatus judges whether or not the test strip 100 is authentic by utilizing the presence or absence of the identifying invisible marker portion 103 on the base member 101. The identifying invisible marker portion 103, which is formed on the base member 100 of the regular test strip, cannot be visually recognized by the naked eye under the visible light. Therefore, even when any person, who does not know the presence of the identifying invisible marker portion 103, intends to imitate the regular test strip, it is impossible to imitate the identifying invisible marker portion 103. That is, even when it is intended to imitate the genuine test strip 100 according to this embodiment, a forged test strip, in which the invisible identifying marker portion is absent, is merely obtained. Accordingly, it is possible to judge whether or not the test strip 100 is authentic easily and accurately.

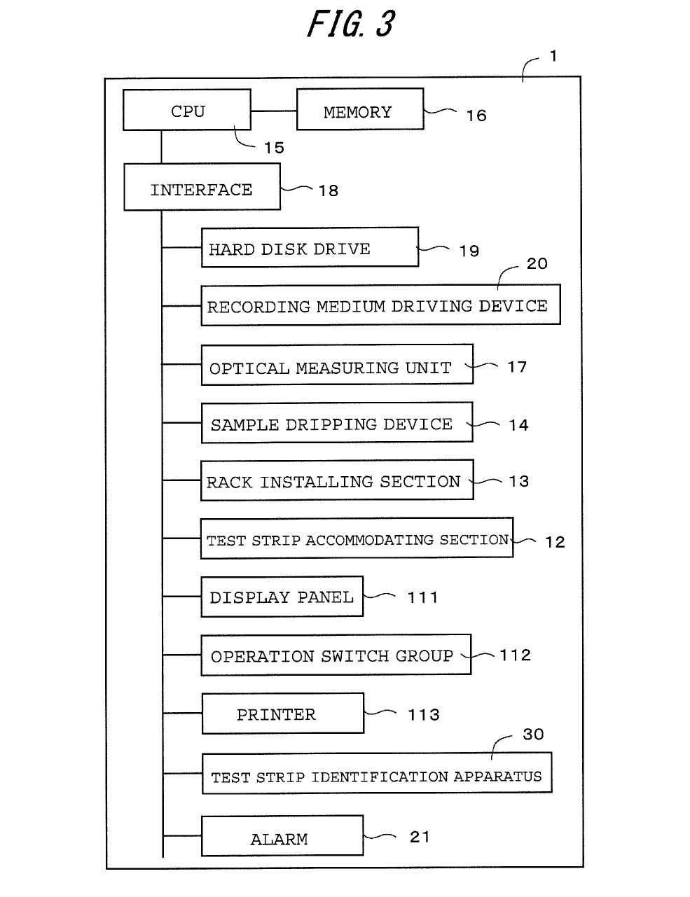

Next, an explanation will be made in detail about the arrangement of the analysis apparatus 1 to which the test strip 100 described above is applied. FIG. 3 shows a structural block diagram illustrating the arrangement of the analysis apparatus 1 according to the first embodiment. An apparatus main body 11 is a casing (housing) for accommodating respective elements for constructing the analysis apparatus 1. The analysis apparatus 1 is a computer which is provided with, for example, the apparatus main body 11, a test strip accommodating section 12, a rack installing section 13, a sample dripping device 14, a central processing unit (hereinafter referred to as “CPU”) 15, a memory 16, an optical measuring unit 17, an interface 18, a hard disk drive (hereinafter referred to as “HDD”) 19, a recording medium driving device 20, an alarm 21, and a test strip identification apparatus 30. The apparatus main body 11 is provided with a display panel 111, an operation switch group 112, and a printer 113.

CPU 15 is the central processing unit which controls the analysis apparatus 1 by executing the computer program. The memory 16 stores the control program (for example, “analysis program” as described later on) which relates to the analysis process for the sample to be executed by CPU 15, and the data which is to be processed by CPU 15. The memory 16 is provided with a volatile random access memory (RAM) and a non-volatile read only memory (ROM). ROM stores, for example, the programs, the parameters, and the various calibration curve data which are necessary for the analysis apparatus 1 to function. RAM provides the work area for CPU 15, and RAM temporarily stores, for example, a part or parts of the program of the operating system (OS) and the application program to be executed by CPU 15. CPU 15 executes various processes in accordance with the programs stored in the memory 16.

The interface 18 connects CPU 15 and various devices, for which it is also allowable to adopt, for example, a serial interface such as the universal serial bus (USB) or the like and a parallel interface such as the peripheral component interconnect (PCI) or the like. CPU 15 and the respective devices are connected to one another by means of the single interface 18. However, CPU 15 and the respective devices may be connected to one another by means of a plurality of different interfaces.

HDD 19 stores various types of data to be used by the analysis apparatus 1, including, for example, various programs to be loaded on RAM of the memory 16. HDD 19 stores various types of data to be processed by CPU 15. Further, for example, OS is installed in HDD 19. The recording medium driving device 20 is, for example, a driving device for the compact disc (CD), the digital versatile disk (DVD), HD-DVD, the blu-ray disk or the like. Alternatively, the recording medium driving device 20 may be an input/output device for a card medium having a non-volatile memory such as a flash memory or the like. The recording medium, which is driven by the recording medium driving device 20, stores, for example, the computer programs, the input data and the like to be installed into HDD 19.

The operation switch group 112 includes various switches which are provided for a user (operator) to operate the analysis apparatus 1. The operation switch group 112 includes, for example, a power source switch which is provided to switch ON/OFF a main power source of the analysis apparatus 1, a measurement start switch which is provided to start the analysis process (measuring process) for the sample, and a printing switch which is provided to print an analysis result of the sample obtained by the analysis apparatus 1 by means of the printer 113.

The display panel 111 is provided with, for example, LCD (liquid crystal display) and a light-emitting diode. The display panel 111 is controlled by CPU 15 to display various types of information. For example, when a measurement start switch, which is included in the operation switch group 112, is depressed, for example, by the user, then the various measurement items as described above are measured for the sample, and an analysis result (examination result), which is obtained on the basis of the measurement result thereof, is displayed on the display panel 111. Other than the above, for example, the data to be process by CPU 15 and the data to be stored in the memory can be displayed on the display panel 111. On the other hand, when the printing switch described above is depressed, the analysis result of the sample is outputted and printed, for example, on the recording paper by means of the printer 113.

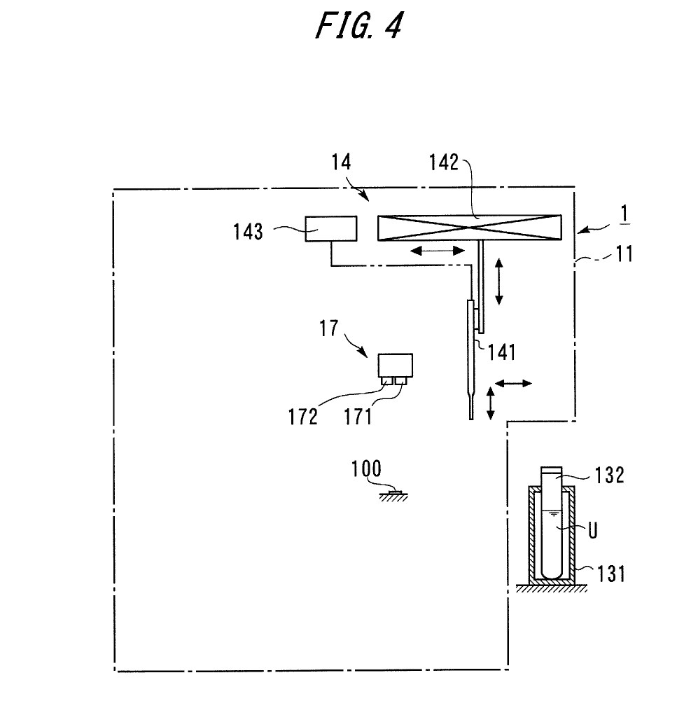

A sample rack 131 shown in FIG. 4 is installed on the rack installing section 13. The sample rack 131 is a rack which is provided to upstandingly hold a plurality of sample containers (sample tubes) 132 in each of which the urine as the sample (indicated by a symbol U in the drawing) is accommodated. The rack installing section 13 has a circulating driving belt 133 which is capable of conveying the sample rack 131 installed on the rack installing section 13 in two directions respectively in the horizontal plane in the rack installing section 13, and a pusher (not shown). The rack installing section 13 also functions as a transport apparatus which conveys (transports) the sample rack 131 along with a constant route by driving the circulating driving belt 133 and the pusher. The rack installing section 13 (also referred to as “sampler” in some cases) can be constructed in the same manner as those having been hitherto known (see, for example, Japanese Patent Application Laid-open No. 2009-229232).

The sample dripping device 14 includes, for example, a nozzle 141, a nozzle driving device 142 which drives the nozzle 141 in the upward-downward direction and the horizontal direction in the apparatus main body 11, and a driving pump 143 which controls, for example, the suction and the discharge (dropping or dropwise addition) of the urine to be performed by the nozzle 141. The nozzle driving device 142 can be constructed, for example, by an actuator or a circulating driving belt.

When the urine is sucked by the nozzle 141, then CPU 15 controls the nozzle driving device 142 to move the nozzle 141 to a position disposed over or above the sample container 132 in which the urine is accommodated, and then the nozzle 141 is moved downwardly. Further, CPU 15 controls the driving pump 143, and the nozzle 141 is allowed to suck the urine contained in the sample container 132.

The test strip 100, which is accommodated in the test strip accommodating section 12, is taken out one by one by means of an unillustrated supply device. The taken out test strip 100 is conveyed (carried or supplied) to a predetermined dripping position. The supply device is controlled by CPU 15. The supply device can be constructed in the same manner as those having been hitherto known (for example, a rotary drum or a conveying or transporting device disclosed in Japanese Patent Application Laid-open No. 2009-229232).

When the urine is dripped onto the test strip 100 by means of the nozzle 141, then the nozzle driving device 142 is controlled by CPU 15, and the nozzle 141 is moved to a position disposed over or above the reagent pad 102 of the test strip 100. After that, the driving pump 143 is controlled by CPU 15, and a predetermined amount of the urine is dripped from the nozzle 141 onto the reagent pad 102. The dripping operation as described above is repeated a number of times corresponding to the number of the reagent pads 102 disposed on the test strip 100, and the dripping process comes to an end. After the dripping process for the sample with respect to the test strip 100 is completed, the nozzle 141 is washed with a washing liquid (for example, distilled water). Although the arrangement for washing the nozzle 141 is not shown, the arrangement may be constructed in the same manner as those having been hitherto known (for example, those described, for example, in Japanese Patent Application Laid-open Nos. 2000-321270 and 2009-229232).

The optical measuring unit 17 is an optical device or instrument which is disposed in the apparatus main body 1. The optical measuring unit 17 is controlled by CPU 15, and thus the coloration state of each of the reagent pads 102 on the test strip 100 is optically measured after carrying out the dripping process.

The optical measuring unit 17 has a light-emitting device 171 which emits the light with respect to the respective reagent pads 102 of the test strip 100 dripped with the sample, and a light-receiving device 172 which receives the reflected light allowed to come from the respective reagent pads 102. The optical measuring unit 17 is the apparatus which is provided to obtain the information corresponding to the degree of the color development (coloration reaction or coloring reaction) of each of the reagent pads 102. The optical measuring unit 17 is provided, for example, in such a form that the optical measuring unit 17 is reciprocatively movable in the longitudinal direction of the test strip 100 on which the sample has been dripped. The light-emitting device 171 is, for example, a semiconductor laser or a light-emitting diode (LED) for emitting the light having a specified peak wavelength. On the other hand, the light-receiving device 172 is, for example, a photodiode for receiving the reflected light from each of the reagent pads 102.

CPU 15 allows the light-emitting device 171 to emit the light onto the reagent pads 102, while moving the optical measuring unit 17 along the array of the respective reagent pads 102 of the test strip 100 (in the longitudinal direction of the test strip 100). The reflected lights, which come from the plurality of reagent pads 102, are successively received by the light-receiving device 172 continuously. CPU 15 acquires the measurement result obtained by the optical measuring unit 17. CPU 15 calculates, for example, the numerical information in relation to the measuring components (specified components) contained in the sample, i.e., the concentrations and the amounts of, for example, hemoglobin, glucose, and protein contained in the urine on the basis of the measurement result acquired from the optical measuring unit 17 and the calibration curve or working curve data stored in ROM of the memory 16.

The calibration curve data is the data in which the relationship between the response measured values obtained by measuring the respective reagent pads 102 by the optical measuring unit 17 and the numerical information in relation to the measuring components corresponding to the reagent pads 102 is stored. The calibration curve data is previously prepared corresponding to each of the measuring components as the calculation objective. The calculation result obtained by CPU 15, i.e., the analysis result of the sample is displayed on the display panel 111. Further, when the printing switch is depressed, for example, by the user, the analysis result of the sample is outputted and printed on the recording paper by means of the printer 113.

Next, an explanation will be made about the test strip identification apparatus 30 carried on the analysis apparatus 1 according to the embodiment of the present invention. It is now premised that the regular test strip is used for the analysis process for the sample in order to secure the reliability of the analysis result obtained by the analysis apparatus 1, for the following reason. That is, if the analysis process is performed for the sample by using any test strip other than the regular test strip, i.e., any forged test strip or the like, the inconvenience tends to arise, for example, such that the analysis error is increased. However, the regular test strip is similar to the forged test strip in appearance in many cases. It is not easy for the user (operator) to apparently distinguish the difference between the both. Therefore, if the user erroneously recognizes the forged test strip as the regular test strip, and the user uses the forged test strip for the analysis process, then any error arises in the analysis result of the sample. It is feared that the user may fail to notice the illness or the disease of an examinee on account thereof. In view of the above, the test strip identification apparatus 30, which performs the identification of the test strip 100, is provided in order to avoid and suppress the circumstance as described above.

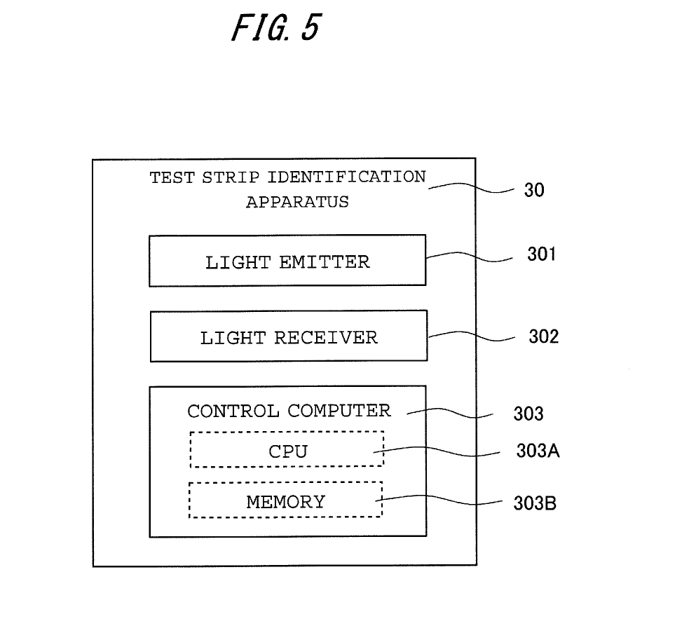

FIG. 5 shows a structural block diagram illustrating a detailed arrangement of the test strip identification apparatus according to the first embodiment. The test strip identification apparatus 30 comprises a light emitter 301, a light receiver 302, and a control computer 303. As described in detail later on, the light emitter 301 emits the light toward the identifying invisible marker portion 103 of the test strip 100 to serve as the identification objective. The light receiver 302 receives the light emitted by the invisible ink (fluorescent ink) of the identifying invisible marker portion 103 exposed with the emitted light emitted by the light emitter 301. The control computer 303 is a microcomputer constructed to include, for example, CPU (central processing unit) 303A and a memory 303B. CPU 303A executes the OS (operating system) program which is the basic software stored in the memory 303B and the application programs which are provided to execute the control of the light emitter 301 and the light receiver 302 and the data processing. The identification program, which is provided to identify the test strip as explained below, is one of the application programs as well.

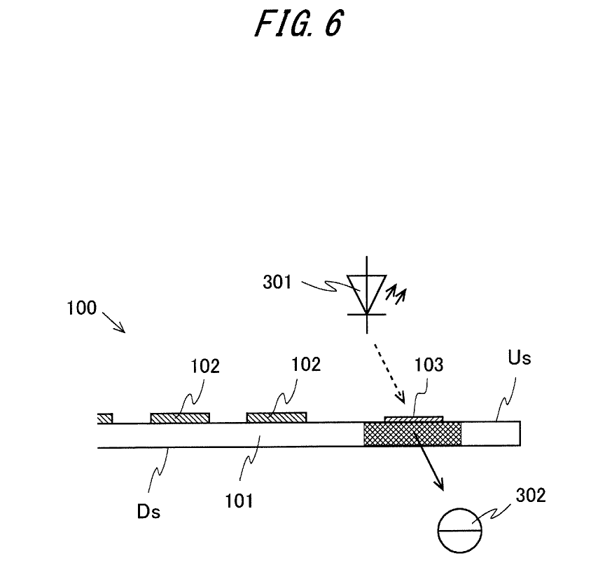

FIG. 6 illustrates the positional relationship between the test strip 100 and the respective devices of the test strip identification apparatus 30 according to the first embodiment. The test strip 100 shown in this drawing is set at the dripping position as described above. Further, the test strip 100 is in a state before the sample is dripped by the sample dripping device 14.

When the test strip 100 is identified by means of the test strip identification apparatus 30, the light emitter 301 and the light receiver 302 are arranged on the side of the front surface Us and on the side of the back surface Ds of the test strip 100 with the test strip 100 intervening therebetween as shown in the drawing. More specifically, the light emitter 301 is arranged over or above the front surface Us of the base member of the test strip 100 in order to emit the emitting light onto the fluorescent ink for forming the identifying invisible marker portion 103. In this embodiment, the light emitter 301 is the light-emitting diode (LED) which emits the ultraviolet ray (ultraviolet light) for exciting the fluorescent ink. However, there is no limitation thereto. On the other hand, the light receiver 302 is the light-receiving element which is arranged on the side opposite to the light emitter 301 with the test strip 100 intervening therebetween as shown in the drawings, i.e., under or below the back surface Ds of the base member of the test strip 100. In this embodiment, the photodiode, which receives the light having the wavelength ranging from the near ultraviolet ray to the near infrared ray, is adopted for the light receiver 302. However, there is no limitation thereto.

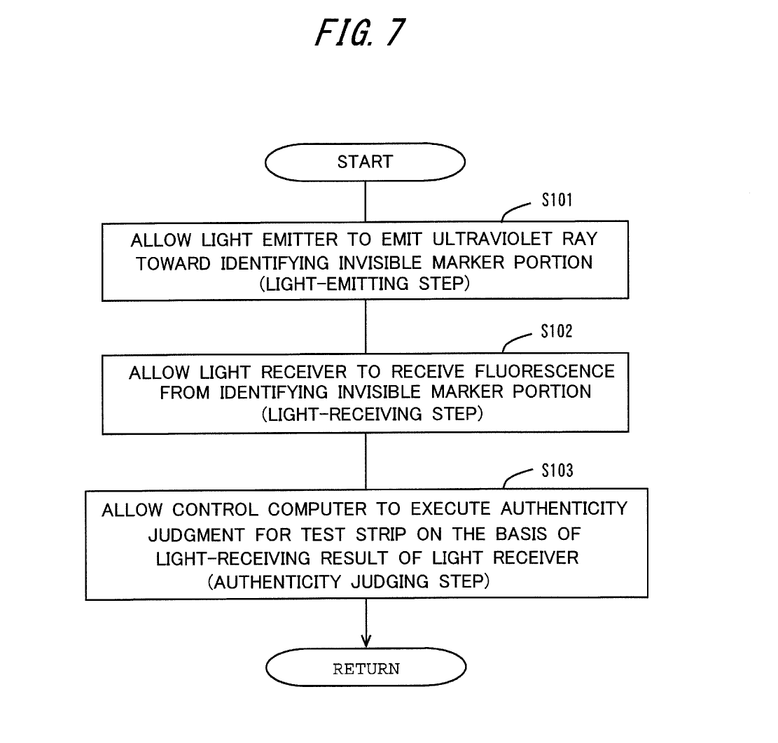

Next, an explanation will be made with reference to FIG. 7 about the specified control contents executed by the test strip identification apparatus 30. FIG. 7 shows a flow chart illustrating the contents of a process flow to be executed by the control computer 303 of the test strip identification apparatus 30. The process flow shown in FIG. 7 is realized by allowing the hardware resources including, for example, CPU 303A and the memory 303B to cooperate with each other, specifically by executing the identification program stored in the memory 303B by CPU 303A. The identification program concerning this process flow is executed by using, for example, the trigger of the fact that the test strip 100, which has been accommodated in the test strip accommodating section 12, is set at the dripping position by means of the unillustrated supply device. Alternatively, the identification program may be realized by allowing the hardware resources including, for example, CPU 15 and the memory 16 to cooperate with each other. In this embodiment, the control computer 303 corresponds to the identification unit according to the present invention.

When the control computer 303 starts the execution of the identification program, the light emitter 301 firstly emits the ultraviolet ray (ultraviolet light) toward the identifying invisible marker portion 103 (marker portion arrangement area Asmk) formed on the front surface Us of the base member of the test strip 100 as the identification objective in Step S101 (light-emitting step). As explained with reference to FIG. 1, the fluorescent ink, which is used for the identifying invisible marker portion 103, is excited by using the ultraviolet ray (ultraviolet light) as the exciting light, and the fluorescence of the visible light is emitted. Subsequently, in Step S102 (light-receiving step), the light receiver 302 receives the fluorescence emitted by the fluorescent ink for forming the identifying invisible marker portion 103 exposed with the ultraviolet ray (ultraviolet light) in Step S101 (light-emitting step).

Step S101 (light-emitting step) and Step S102 (light-receiving step) described above will be explained in detail. When the test strip, which is irradiated with the ultraviolet ray by the light emitter 301, is the regular test strip, the fluorescent ink, which is exposed with the ultraviolet ray, emits the fluorescence of the visible light. In this case, the marker portion arrangement area Asmk and the margin area Asmg, which are included in the base member 101 of the test strip 100 and which are arranged in the in-plane direction thereof, are formed of the ultraviolet ray-intransmissive and fluorescence-transmissive type material. Therefore, the fluorescence, which is emitted by the fluorescent ink excited by the ultraviolet ray, is transmitted through the base member 101 toward the back surface Ds of the base member. As a result, the light receiver 302 can receive the fluorescence transmitted through the base member 101 on the side of the back surface Ds of the base member. For example, the content of the fluorescent dye and the material amount of the fluorescent ink as the invisible substance used for the identifying invisible marker portion 103 are set to be inappropriate amounts to emit the fluorescence to such an extent that the fluorescence signal is reliably detected by the light receiver 302 when the fluorescent ink is exposed with the ultraviolet ray emitted from the light emitter 301.

On the other hand, if the test strip 100, which is set at the dripping position, is not the genuine product, and the test strip 100 is, for example, any forged test strip, then the invisible identifying marker portion 103 is absent on the front surface Us of the base member 101. In this case, even when the light emitter 301 emits the ultraviolet ray in the light-emitting step toward the portion of the front surface Us of the base member 101 at which the identifying invisible marker portion 103 may be present in the case of the regular test strip, i.e., toward the marker portion arrangement area Asmk, the fluorescence is not emitted, because the fluorescent ink, which uses the ultraviolet ray as the exciting light, is absent. The ultraviolet ray, which is emitted by the light emitter 301, is not transmitted through the base member of the forged test strip. Therefore, the ultraviolet ray is not received by the light receiver 302. As for the margin area Asmg, the size and the range thereof may be determined so that the ultraviolet ray, which is included in the ultraviolet ray emitted from the light emitter 301 and which is transmitted through the outer area of the base member 101 disposed outside the margin area Asmg, is not received by the light receiver 302. Various materials, which reflect the ultraviolet ray and/or which absorb the ultraviolet ray, can be applied as the base member 101 through which the ultraviolet ray is not transmitted.

In Step S103 (authenticity judging step), the test strip 100 is identified by judging whether or not the test strip 100 is authentic, on the basis of the light-receiving result of the light receiver 302 in Step S102 (light-receiving step) by means of the control computer 303. Specifically, the control computer 303 judges that the test strip 100 is the regular test strip if the light receiver 302 receives the fluorescence in Step S102. The control computer 303 judges that the test strip 100 is any forged test strip if the fluorescence is not received. When the process of this step comes to an end, the identification program executed by CPU 303A comes to an end. The authenticity judging step can be recognized as the identifying step.

As described above, according to the test strip 100 of this embodiment, the identifying invisible marker portion 103 is formed of the invisible substance. Therefore, the invisible identifying marker portion 103 can be preferably used for the authenticity judgment performed by the test strip identification apparatus 30. Further, it is possible to judge whether or not the test strip is authentic, more conveniently and accurately.

In this embodiment, the identification result of the test strip 100, which is obtained by the test strip identification apparatus 30, is inputted into CPU 15 of the analysis apparatus 1. CPU 15 stores the identification result acquired from the test strip identification apparatus 30 in HDD 19. When CPU 15 recognizes the fact that the test strip 100, which is set at the dripping position at present, is the forged test strip on the basis of the identification result, CPU 15 may inform the user of the fact. CPU 15 may inform the user of the fact by means of the sound or voice by operating the alarm 21, or CPU 15 may display the fact as letter information on the display panel 111.

The analysis apparatus 1 performs the analysis process for the sample by using the test strip 100 identified by the test strip identification apparatus 30. The identified test strip 100, which is referred to herein, is the test strip which is identified as the genuine product. That is, the dripping process with the sample by means of the sample dripping device 14 and the measuring process by using the optical technique by means of the optical measuring unit 17 are performed with respect to the regular test strip 100. CPU 15 analyzes the sample on the basis of the measurement result thereof.

On the other hand, the sample dripping process is not performed by the sample dripping device 14 in relation to the test strip which is identified as an imitation by the test strip identification apparatus 30. The test strip, which is identified as the imitation, may be abandoned or discarded to a waste container (not shown) by means of an unillustrated transport apparatus. In this case, another test strip 100, which is accommodated in the test strip accommodating section 12, is transported or conveyed to the dripping position, and the new test strip 100 is appropriately identified by the test strip identification apparatus 30. In this way, it is possible to avoid the execution of the sample analysis process by using any forged test strip. It is possible to suppress the increase in the analysis error. Therefore, the reliability of the analysis result of the sample is not lowered as well. Further, for example, it is possible to more reliably avoid such an inconvenience that the illness or the disease of the examinee is not found and noticed.

In this embodiment, the control, which relates to the identification of the test strip 100, is performed by the exclusive computer (control computer 303) provided for the test strip identification apparatus 30. However, this function may be realized by CPU 15 and the memory 16 of the analysis apparatus 1. In other words, the control, which relates to the identification of the test strip 100, may be performed by CPU 15 and the memory 16.

In this embodiment, the identification method for identifying the analysis instrument and the contents of the identification program as the computer program to be executed by the computer (control computer 303 of the test strip identification apparatus 30) for identifying the analysis instrument are those explained in relation to the process flow with reference to FIG. 7.

Further, the medium, in which the identification program is recorded on any recording medium capable of being read by the computer, also belongs to the category of the present invention. As for the recording medium on which the identification program is recorded, the test strip can be identified, for example, by allowing the control computer 303 of the test strip identification apparatus 30 to read and execute the program of the recording medium. The recording medium, which is readable or capable of being read by the computer, herein refers to the recording medium on which the information such as the data, the program or the like can be accumulated by means of the electric, magnetic, optical, mechanical, or chemical action and the information can be read from the computer. The recording medium, which is included in those as described above and which is removable from the computer, includes, for example, the flexible disk, the magneto-optical disk, CD-ROM, CD-R/W, DVD, DAT, the 8 mm tape, and the memory card. On the other hand, the recording medium, which is fixed to the computer, includes, for example, the hard disk and ROM (read only memory).

### First Modified Embodiment

Next, a first modified embodiment will be explained. At first, the test strip 100, which has been explained with reference to FIG. 1, is provided with the identifying invisible marker portion 103 formed on the front surface Us of the base member. However, the identifying invisible marker portion 103 may be provided on the back surface Ds of the base member. In this arrangement, the light emitter 301 of the test strip identification apparatus 30 is arranged under or below the back surface Ds of the base member 101, and the light receiver 302 is arranged over or above the front surface Us of the base member 101. That is, the arrangement relationship, which relates to the light emitter 301 and the light receiver 302 with respect to the test strip 100 shown in FIG. 6, is mutually exchanged.

### Second Modified Embodiment

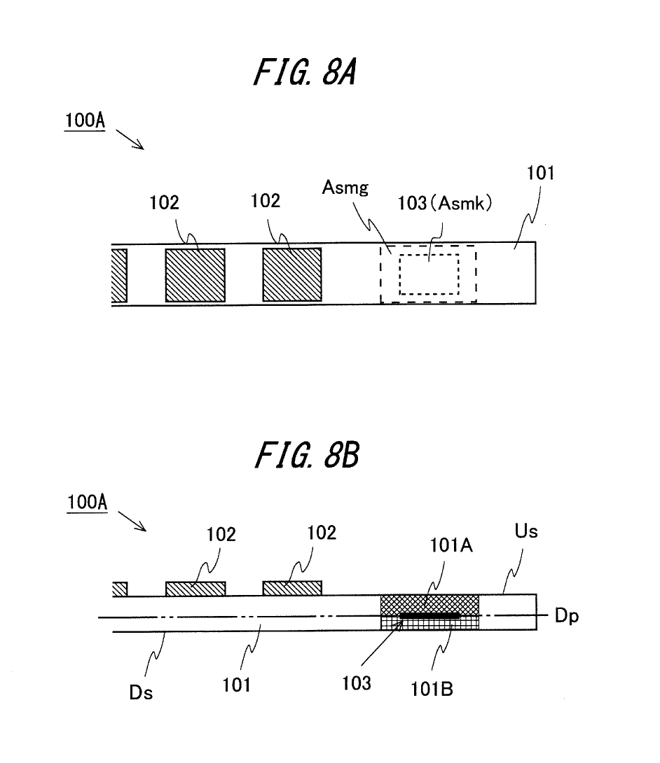

FIG. 8 shows an arrangement of a test strip 100A according to a second modified embodiment. FIG. 8A shows a front surface Us of a base member 101 of the test strip 100A, and FIG. 8B shows a sectional view taken along the longitudinal direction of the test strip 100A. In these drawings, the components or parts, which are the same as those of the test strip 100 shown in FIG. 1, are designated by the common symbols or reference numerals, and thus any detailed explanation thereof will be omitted. The test strip 100A is different from the test strip 100 in that the identifying invisible marker portion 103 is provided by being embedded in the base member 101.

With reference to this drawing, the marker portion arrangement area Asmk is an area in which the identifying invisible marker portion 103 is embedded in the base member 101 in the in-plane direction of the base member 101. As for the test strip 100A, a portion, which is disposed on the side of one surface of the front surface Us of the base member 101 and the back surface Ds of the base member 101, is formed of a material (hereinafter referred to as “ultraviolet ray-transmissive material”) through which the ultraviolet ray is transmitted, and a portion, which is disposed on the side of the other surface, is formed of an ultraviolet ray-intransmissive and fluorescence-transmissive type material, with the boundary of the embedding position (hereinafter referred to as “marker portion embedding depth”) Dp of the identifying invisible marker portion 103 in the thickness direction of the base member 101 in at least the marker portion arrangement area Asmk of the base member 101 (in this case, for example, the marker portion arrangement area Asmk and the predetermined margin area Asmg to surround the same). In FIG. 8B, the portion, which ranges from the marker portion embedding depth Dp to the front surface Us of the base member 101, is formed by using the ultraviolet ray-intransmissive and fluorescence-transmissive type material (indicated by a symbol or reference numeral 101A in the drawing), and the portion, which ranges from the marker portion embedding depth Dp to the back surface Ds of the base member 101, is formed by using the ultraviolet ray-transmissive material (indicated by a symbol or reference numeral 101B in the drawing). However, the respective materials may be mutually exchanged with each other.

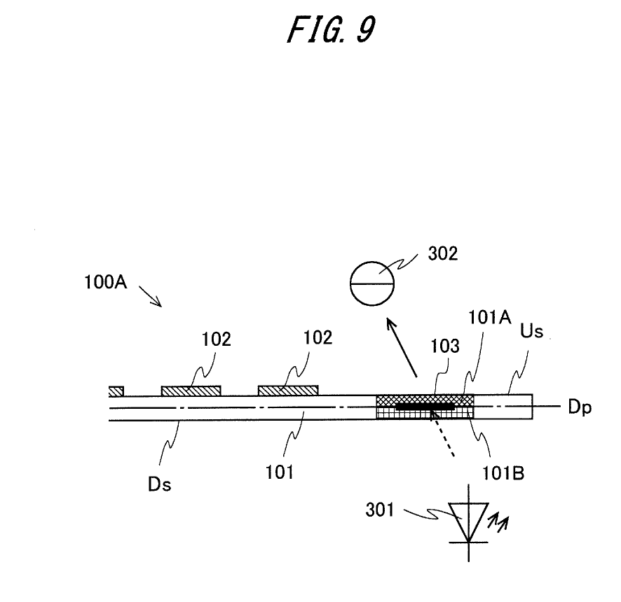

FIG. 9 illustrates the positional relationship between the test strip 100A and the respective devices of the test strip identification apparatus 30 according to the second modified embodiment. In this drawing, the components or parts, which are the same as those shown in FIG. 6, are designated by the common symbols or reference numerals. As shown in FIG. 9, the light receiver 302 is arranged over or above the front surface Us of the base member 101 of the test strip 100A, and the light emitter 301 is arranged under or below the back surface Ds of the base member 101.

According to the test strip identification apparatus 30 constructed as described above, when it is judged whether or not the test strip 100A is authentic, the light emitter 301 emits the ultraviolet ray toward the marker portion arrangement area Asmk in which the identifying invisible marker portion 103 is embedded (light-emitting step). The ultraviolet ray is transmitted through the ultraviolet ray-transmissive material 101B for constructing the base member 101. In this case, if the test strip 100A is the regular test strip, then the ultraviolet ray arrives at the identifying invisible marker portion 103 embedded at the marker portion embedding depth Dp, and thus the fluorescent ink, which forms the identifying invisible marker portion 103, is excited. As a result, the fluorescence, which is emitted from the fluorescent ink, travels toward the front surface Us of the base member 101 while being transmitted through the ultraviolet ray-intransmissive and fluorescence-transmissive type material 101A. The fluorescence allowed to arrive at the front surface Us of the base member 101, i.e., the fluorescence transmitted through the ultraviolet ray-intransmissive and fluorescence-transmissive type material 101A is received by the light receiver 302 on the side of the front surface Us of the base member 101 (light-receiving step).

On the other hand, if the test strip 100A is any forged test strip, even when the light emitter 301 emits the ultraviolet ray toward the marker portion arrangement area Asmk in the light-emitting step, then the fluorescence is not emitted, although the ultraviolet ray arrives at the marker portion embedding depth Dp from the back surface Ds of the base member 101, because the invisible identifying marker portion 103 is absent at the position of the marker portion embedding depth Dp. Further, the ultraviolet ray, which is emitted by the light emitter 301, is not transmitted through the ultraviolet ray-intransmissive and fluorescence-transmissive type material 101A. Therefore, the ultraviolet ray is not received by the light receiver 302. For example, the size and the range of the margin area Asmg are determined so that the ultraviolet ray, which is included in the ultraviolet ray emitted from the light emitter 301 and which is transmitted through the outer area disposed outside the margin area Asmg, is not received by the light receiver 302.

The control computer 303 (identification unit) identifies the test strip 100A on the basis of the light-receiving result obtained by the light receiver 302 (identifying step). That is, when the fluorescence is received by the light receiver 302, the control computer 303 can judge that the test strip 100A is the regular test strip. When the fluorescence is not received by the light receiver 302, the control computer 303 can judge that the test strip 100A is any forged test strip. The method for utilizing the identification result of the test strip brought about by the test strip identification apparatus 30 has been described, and hence any explanation thereof is omitted.

### Second Embodiment

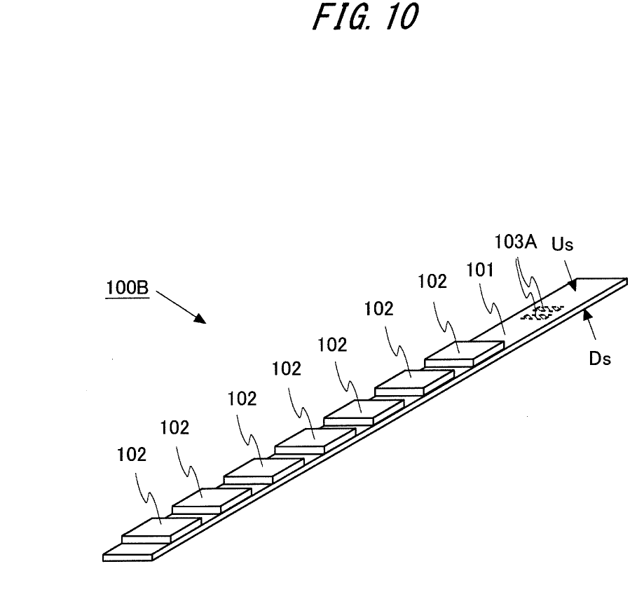

FIG. 10 shows a schematic perspective view illustrating a test strip 100B according to a second embodiment. The hardware construction, which is provided for the analysis apparatus for applying the test strip 100B thereto, is common to that of the first embodiment. The test strip 100B has the base member 101 and the reagent pads 102 which are the same as or equivalent to those of the test strip 100 shown in FIG. 1. The symbol or reference numeral 103A shown in FIG. 10 indicates an identifying invisible marker portion of this embodiment. The identifying invisible marker portion 103A is also the same as or equivalent to the identifying invisible marker portion 103 shown in FIG. 1 in that the identifying invisible marker portion 103A is formed on the front surface Us of the base member 101 by using the fluorescent ink as exemplified as the invisible substance by way of example.

The identifying invisible marker portion 103A is a bar code which is formed by using a fluorescent ink that is invisible (visually unrecognizable) with the naked eye under the visible light. The identifying invisible marker portion 103A is an identifier or identification element which expresses the numerical value and/or the letter information by means of the thickness of the striped pattern-shaped line depicted with the fluorescent ink. The identification information of the test strip 100B is the information to identify the test strip 100B. The identification information includes, for example, the product specification information of the test strip 100B (hereinafter referred to as “test strip specification information”) and the analysis-related information to be used when the analysis process is performed for a sample by means of the analysis apparatus 1 by using the test strip 100B. The test strip specification information may also include the information about, for example, the term of validity of the test strip 100B. The test strip specification information is the information which relates to the specification of the test strip 100B, including, for example, the production lot and the manufacturer's serial number of the test strip. The analysis-related information includes, for example, the type of the sample to serve as the analysis objective, the types of the measuring components (specified components), and the identification marks (hereinafter referred to as “calibration curve data identification marks”) corresponding to the respective pieces of the calibration curve data stored in ROM of the memory 16. The calibration curve data identification marks are appended, for example, as the marks which differ depending on the respective types or classifications of the measuring components of the sample.

The arrangement relationship between the test strip 100B and the respective devices of the test strip identification apparatus 30, which is provided when the identification process is performed for the test strip 100B, is the same as or equivalent to the exemplary arrangement shown in FIG. 6 (the symbol or reference numeral of the test strip 100 shown in FIG. 6 is exchanged with that of the test strip 100B). That is, the light emitter 301 is arranged on the side of the front surface Us of the base member 101 of the test strip 100B, and the light receiver 302 is arranged on the side of the back surface Ds of the base member 101 of the test strip 100B.

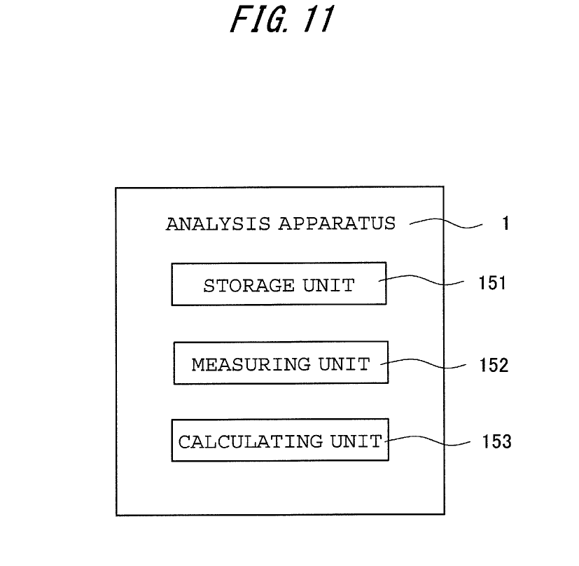

Next, an explanation will be made about the analysis process for the sample performed by the analysis apparatus 1. FIG. 11 shows a functional block diagram illustrating the analysis apparatus 1 according to the second embodiment. The analysis apparatus 1 functions as a computer comprising a storage unit 151 which acquires, from the test strip identification apparatus 30, the identification information (test strip specification information, analysis-related information) stored in the identifying invisible marker portion 103A of the test strip 100B and which stores the acquired analysis-related information, a measuring unit 152 which measures the sample with which the reagent pads 102 of the test strip 100B are impregnated, and a calculating unit 153 which calculates the numerical information including, for example, the concentrations in relation to the measuring components of the sample on the basis of the measurement result of the sample measured by the measuring unit 152 and the analysis-related information stored in the storage unit 151, by allowing CPU 15 to interpret and execute the analysis program developed or expanded on RAM or ROM of the memory 16.

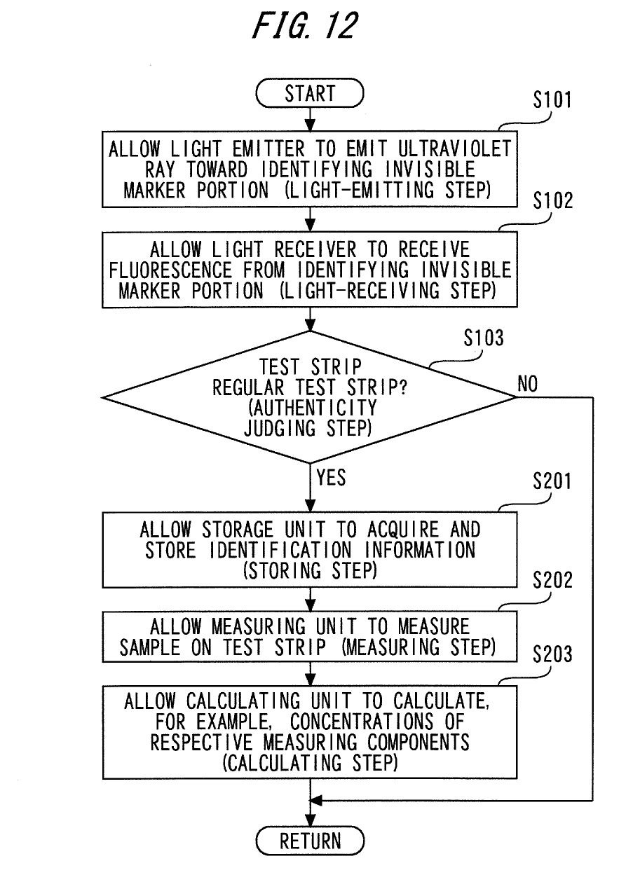

FIG. 12 shows a flow chart illustrating the contents of a process flow to be executed by the analysis apparatus 1 according to the second embodiment. The process flow shown in FIG. 12 is realized by allowing CPU 15 to execute the analysis program stored, for example, in the memory 16 while allowing the hardware resources including, for example, CPU 15, the memory 16, and the test strip identification apparatus 30 to cooperate with each other. The step numbers, which are the same as those shown in FIG. 7, mean the fact that the process contents thereof are the same, and any detailed explanation of the process contents will be omitted.

When the analysis program is executed by CPU 15 of the analysis apparatus 1, the light emitter 301 firstly emits the ultraviolet ray toward the identifying invisible marker portion 103A (marker portion arrangement area Asmk) formed on the front surface Us of the base member of the test strip 100B in Step S101 (light-emitting step). Subsequently, in Step S102 (light-receiving step), the light receiver 302 receives the light emitted by the fluorescent ink which forms the identifying invisible marker portion 103A and which is exposed with the ultraviolet ray. The light receiver 302 converts the received light into the electric signal, and the response signal is outputted to the control computer 303.

In Step S103 (authenticity judging step), the control computer 303 (identification unit) identifies the test strip 100B by executing the authenticity judgment for the test strip 100B on the basis of the light-receiving result of the light receiver 302 obtained in Step S102 (light-receiving step). In other words, it is discriminated whether or not the test strip 100B is the regular test strip, on the basis of the light-receiving result of the light receiver 302. If the light receiver 302 receives the fluorescence in Step S102, then it is discriminated that the test strip 100B is the regular test strip (S103: Yes), and the routine proceeds to Step S201. On the other hand, if the fluorescence is not received by the light receiver 302, it is discriminated that the test strip 100B is any forged test strip (S103: No). If it is discriminated that the test strip 100B is the regular test strip, the control computer 303 identifies the identification information of the test strip 100B on the basis of the light-receiving result obtained by the light receiver 302. If it is discriminated that the test strip 100B is any forged test strip, then the user is informed of that fact, and then the execution of the program is completed. In this embodiment, when the analysis process is performed for the sample, it is judged whether or not the sample is authentic, before dripping the sample onto the test strip 100B. The respective measuring components are measured for the test strip which has been confirmed to be the regular product.

In Step S201 (storing step), the storage unit 151 acquires the identification information of the test strip 100B identified by the control computer 303 (identification unit), and the identification information is stored. In Step S202 (measuring step), the measuring unit 152 acquires the types of the respective measuring components on the test strip 100B, and the sample, with which the reagent pads 102 of the test strip 100B are impregnated, is measured by controlling the respective devices (for example, the sample dripping device 14 and the optical measuring unit 17) on the basis of the information.

In Step S203 (calculating step), the calculating unit 153 acquires the calibration curve data identification mark included in the analysis-related information stored by the storage unit 151. The calculating unit 153 acquires the response measured values corresponding to the respective reagent pads 102 from the optical measuring unit 17. The calculating unit 153 calculates, for example, the concentrations of the respective measuring components (numerical information in relation to the measuring components) on the basis of the calibration curve data corresponding to the response measured values. The calculating unit 153 reads, from the memory 16, the calibration curve data corresponding to the types of the measuring components. When the process of this step comes to an end, the execution of the analysis program comes to an end.

In this embodiment, the identifying invisible marker portion 103A is formed on the front surface Us of the base member 101 of the test strip 100B. However, the identifying invisible marker portion 103A may be formed on the back surface Ds of the base member 101, or the identifying invisible marker portion 103A may be arranged while being embedded in the base member 101, in accordance with the viewpoints which are the same as or equivalent to those of the first embodiment and the modified embodiments thereof. Further, the identification information of the test strip is not limited to the embodiment in which the identification information of the test strip is recorded by using the invisible substance on the identifying invisible marker portion 103. Other than the above, for example, the identification information of the test strip may be recorded on a bar code formed by using a visible ink (for example, a black ink). Such a visible bar code may be arranged on any one of the front surface Us of the base member 101 and the back surface Ds of the base member 101. In the embodiment in which the identification information of the test strip is recorded on the visible bar code, the identifying invisible marker portion, which is to be utilized for the authenticity judgment, is provided distinctly from the visible bar code, for the following reason. That is, the information, which is provided for performing the authenticity judgment to judge whether or not the test strip is the genuine product, cannot be recorded on the visible bar code. Thus, the authenticity judgment is performed for the test strip by utilizing the identifying invisible marker portion formed with the fluorescent ink, while the identification information, which is recorded on the visible bar code, can be utilized to acquire, for example, the information in relation to the specification of the test strip and the calibration curve data identification mark to be used for the analysis process for the sample.

### Third Modified Embodiment

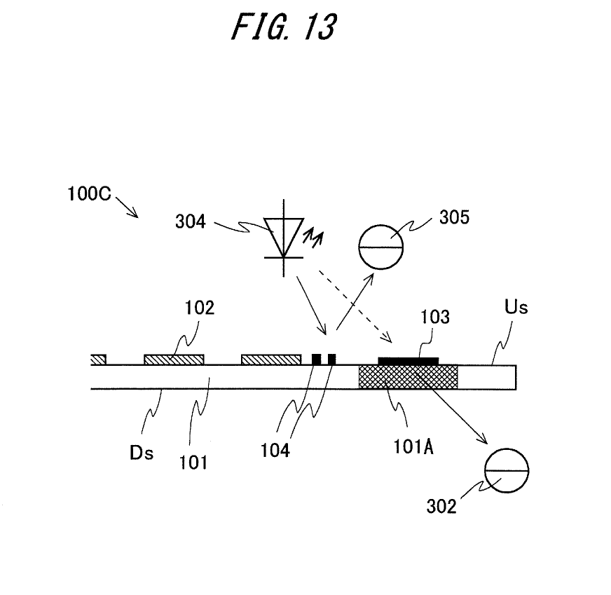

FIG. 13 illustrates the positional relationship between a test strip 100C and respective devices of a test strip identification apparatus 30 according to a third modified embodiment. The test strip 100C has a base member 101 and reagent pads 102 which are the same as or equivalent to those of the test strip 100 shown in FIG. 1. An identifying invisible marker portion 103 is formed on the front surface Us of the base member 101. Further, a visible identification code portion 104 is formed adjacently to the identifying invisible marker portion 103 on the front surface Us of the base member 101. The visible identification code portion 104 is a bar code formed by using a visible ink, on which the identification information of the test strip 100C is recorded.

In this modified embodiment, those provided as optical devices of the test strip identification apparatus 30 include a second light emitter 304, a light receiver 302, and a second light receiver 305. The light receiver 302 is a light-receiving element (for example, a photodiode) which receives the light having the wavelength ranging from the near ultraviolet ray to the near infrared ray as explained with reference to FIG. 6. The second light receiver 305 is also a light-receiving element which is the same as or equivalent to the light receiver 302. The second light emitter 304 is a light-emitting diode (so-called multi LED) which is capable of emitting the ultraviolet ray and the visible light while switching them. The second light emitter 304 and the second light receiver 305 are arranged on the side of the front surface Us of the base member 101 of the test strip 100C. The light receiver 302 is arranged on the side of the back surface Ds of the base member 101 of the test strip 100C.

In the arrangement shown in the drawing, the authenticity judgment in relation to the test strip 100C and the reading of the identification information recorded on the visible identification code portion 104 are performed on the basis of light emission performed by the second light emitter 304 and the light-receiving results obtained by the light receiver 302 and the second light receiver 305. When it is judged whether or not the test strip 100C is authentic, the control computer 303 (identification unit) emits the ultraviolet ray from the second light emitter 304 toward the identifying invisible marker portion 103. If the test strip 100C is the regular test strip, the fluorescent ink of the identifying invisible marker portion 103 emits the fluorescence. Therefore, the fluorescence, which is transmitted through the base member 101 (ultraviolet ray-intransmissive and fluorescence-transmissive type material 101A) to arrive at the back surface Ds of the base member 101, is received by the light receiver 302. Therefore, the control computer 303 can preferably judge whether or not the test strip 100C is authentic, on the basis of the light-receiving result obtained by the light receiver 302.

On the other hand, when the identification information, which is recorded on the visible identification code portion 104, is read, then the control computer 303 emits the visible light from the second light emitter 304 toward the visible identification code portion 104, and the reflected light, which is allowed to come from the visible identification code portion 104, is received by the second light receiver 305. The second light receiver 305 converts the received light into the electric signal, and the response signal is outputted to the control computer 303. The control computer 303 reads the identification information of the test strip 100C on the basis of the response signal acquired from the second light receiver 305. The identification information of the test strip 100C, which is acquired by the control computer 303, is utilized when the analysis process is performed for the sample as explained with reference to the process flow shown in FIG. 12.

### Fourth Modified Embodiment

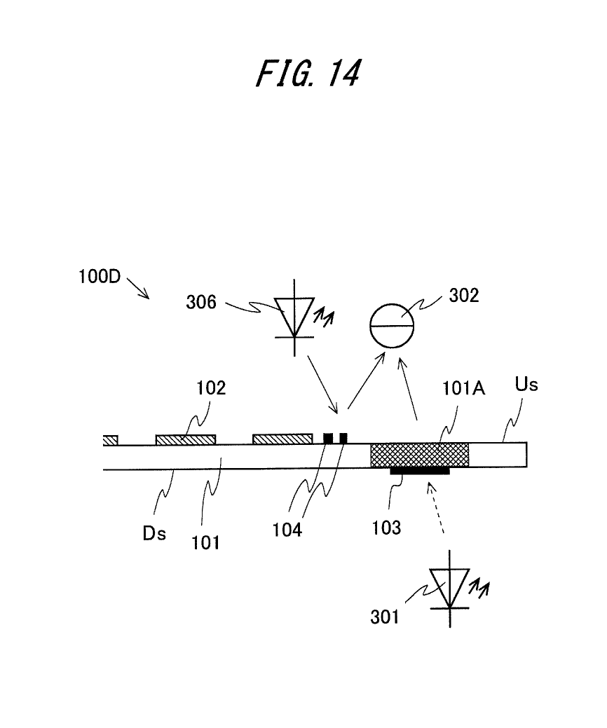

FIG. 14 illustrates the positional relationship between a test strip 100D and respective devices of a test strip identification apparatus 30 according to a fourth modified embodiment. The test strip 100D is the same as or equivalent to the test strip 100C shown in FIG. 13 except that the identifying invisible marker portion 103 is formed on the back surface Ds of the base member. Those provided as the optical system of the test strip identification apparatus 30 include a light emitter 301, a third light emitter 306, and a light receiver 302. The light emitter 301 and the light receiver 302 are as described above. The third light emitter is a light-emitting diode which is capable of emitting the visible light. As shown in the drawing, the third light emitter 306 and the light receiver 302 are arranged on the side of the front surface Us of the base member 101 of the test strip 100D, and the light emitter 301 is arranged on the side of the back surface Ds of the base member 101 of the test strip 100D.

In this exemplary arrangement, the control computer 303 performs the authenticity judgment in relation to the test strip 100D and the reading of the identification information recorded on the visible identification code portion 104 by using the light emitter 301, the third light emitter 306, and the light receiver 302. When it is judged whether or not the test strip 100D is authentic, the control computer 303 emits the ultraviolet ray from the light emitter 301 toward the identifying invisible marker portion 103 formed on the back surface Ds of the base member 101. If the test strip 100D is the regular test strip, the fluorescent ink of the identifying invisible marker portion 103 emits the fluorescence. Therefore, the fluorescence, which is transmitted through the base member 101 (ultraviolet ray-intransmissive and fluorescence-transmissive type material 101A) to arrive at the front surface Us of the base member 101, is received by the light receiver 302. On the other hand, if the test strip 100D is any forged test strip, the fluorescence is not received by the light receiver 302. Therefore, the control computer 303 can preferably judge whether or not the test strip 100D is authentic, on the basis of the light-receiving result obtained by the light receiver 302.

On the other hand, when the identification information, which is recorded on the visible identification code portion 104, is read, then the control computer 303 emits the visible light from the third light emitter 306 toward the visible identification code portion 104, and the reflected light therefrom is received by the light receiver 302. The light receiver 302 converts the received light into the electric signal, and the response signal is outputted to the control computer 303. The control computer 303 can read the identification information of the test strip 100D on the basis of the response signal acquired from the light receiver 302.

In the arrangement shown in FIG. 13, the light source can be used for both of the processes of the authenticity judgment for the test strip and the reading of the identification information. In the arrangement shown in FIG. 14, the light-receiving element can be used in relation to both of the processes as described above. Therefore, these arrangements are effective in view of the realization of the compact apparatus and the reduction of the production cost. From the same or equivalent viewpoint, the optical instruments or devices shown in FIGS. 13 and 14 can be also appropriately used for both of the light-emitting device 171 and the light-receiving device 172 provided for the optical measuring unit 17. However, it is also allowable to perform the authenticity judgment for the test strip and the reading of the identification information by utilizing light sources and light-receiving elements which are independent from each other.

The present invention can be also comprehended as an analysis system for performing the analysis process for a sample by executing the process flow shown in FIG. 12. The analysis program explained in the second embodiment and the medium in which the program is recorded on the recording medium capable of being read by the computer also belong to the category of the present invention. The recording medium, which is readable or capable of being read by the computer, herein refers to the recording medium on which the information such as the data and the program can be accumulated by means of the electrical, magnetic, optical, mechanical, or chemical action and the information can be read from the computer. The recording medium, which is included in those as described above and which is removable from the computer, includes, for example, the flexible disk, the magneto-optical disk, CD-ROM, CD-R/W, DVD, DAT, the 8 mm tape, and the memory card. On the other hand, the recording medium, which is fixed to the computer, includes, for example, the hard disk and ROM (read only memory).

The embodiments of the present invention have been explained above. However, the analysis tool (analysis kit, analysis instrument), the identification apparatus, the analysis apparatus, the analysis system, the identification method, the analysis method, the program, and the recording medium according to the present invention are not limited thereto. The present invention can include combinations thereof as far as possible. The embodiments described above are merely examples to explain the present invention. It is possible to apply various changes to the embodiments described above within a scope without deviating from the gist or essential characteristics of the present invention.

What is claimed is:

1. An analysis tool having a reagent portion to be used for an analysis process for a sample, the analysis tool comprising:
an identifying marker portion which is formed by using an invisible substance.

2. The analysis tool according to claim 1, wherein the identifying marker portion is formed by using a fluorescent substance which emits fluorescence by using an ultraviolet ray as an exciting light.

3. The analysis tool according to claim 2, wherein:
the identifying marker portion is provided on a front surface or a back surface of a base member; and
at least an area of the base member, in which the identifying marker portion is provided, is formed of a material through which the ultraviolet ray is not transmitted and through which the fluorescence emitted by the fluorescent substance is transmitted.

4. The analysis tool according to claim 2, wherein:
the identifying marker portion is provided while being embedded in a base member; and
a portion disposed on a side of one surface is formed of a material through which the ultraviolet ray is transmitted and a portion disposed on a side of the other surface is formed of a material through which the ultraviolet ray is not transmitted and through which the fluorescence emitted by the fluorescent substance is transmitted, with a boundary disposed at an embedding position of the identifying marker portion in a thickness direction of the base member in at least an area of the base member in which the identifying marker portion is embedded.

5. The analysis tool according to claim 1, wherein identification information about the analysis tool is recorded by using the invisible substance on at least a part of the identifying marker portion.

6. The analysis tool according to claim 5, wherein the identification information includes at least any one of product specification information about the analysis tool and analysis-related information to be used when the analysis process is performed for the sample.

7. An identification apparatus for identifying an analysis tool having a reagent portion to be used for an analysis process for a sample, wherein:
the analysis tool further includes an identifying marker portion which is formed by using an invisible substance, the identification apparatus comprising:
a light emitter which emits a light toward the identifying marker portion;
a light receiver which receives a light emitted by the invisible substance for forming the identifying marker portion exposed with the emitted light emitted by the light emitter; and
an identification unit which identifies the analysis tool on the basis of a light-receiving result obtained by the light receiver.

8. The identification apparatus according to claim 7, wherein:
the identifying marker portion is formed of a fluorescent substance which emits fluorescence by using an ultraviolet ray as an exciting light;
the light emitter emits the ultraviolet ray; and
the light receiver receives the fluorescence emitted by the fluorescent substance for forming the identifying marker portion.

9. The identification apparatus according to claim 8, wherein:
the identifying marker portion is provided on a front surface of a base member of the analysis instrument;
at least an area of the base member, in which the identifying marker portion is provided, is formed of a material through which the ultraviolet ray is not transmitted and through which the fluorescence emitted by the fluorescent substance is transmitted;
the light emitter emits the ultraviolet ray from a side of the front surface of the base member; and
the light receiver receives the fluorescence transmitted through the base member on a side of a back surface of the base member.

10. The identification apparatus according to claim 8, wherein:
the identifying marker portion is provided while being embedded in a base member of the analysis instrument;
a portion disposed on a side of one surface is formed of a material through which the ultraviolet ray is transmitted and a portion disposed on a side of the other surface is formed of a material through which the ultraviolet ray is not transmitted and through which the fluorescence emitted by the fluorescent substance is transmitted, with a boundary disposed at an embedding position of the identifying marker portion in a thickness direction of the base member in at least an area of the base member in which the identifying marker portion is embedded;
the light emitter emits the ultraviolet ray from the side of the one surface of the base member; and
the light receiver receives the fluorescence transmitted through the base member on the side of the other surface of the base member.

11. The identification apparatus according to claim 7, wherein identification information about the analysis tool is recorded by using the invisible substance on at least a part of the identifying marker portion.

12. The identification apparatus according to claim 11, wherein the identification information includes at least any one of product specification information about the analysis tool and analysis-related information to be used when the analysis process is performed for the sample.

13. An analysis apparatus comprising the identification apparatus as defined in claim 7, wherein the analysis process is performed for the sample by using the analysis instrument identified by the identification apparatus.

14. An analysis apparatus for performing an analysis process for a sample by using an analysis tool having a reagent portion, wherein:
the analysis instrument has an identifying marker portion which is formed by using an invisible substance, the identifying marker portion having at least a part thereof on which analysis-related information to be used when the analysis process is performed for the sample is recorded, and the analysis apparatus comprising:
a light emitter which emits a light toward the identifying marker portion;
a light receiver which receives a light emitted by the invisible substance for forming the identifying marker portion exposed with the emitted light emitted by the light emitter;
an identification unit which identifies the analysis tool on the basis of a light-receiving result obtained by the light receiver;
a storage unit which acquires the analysis-related information on the basis of the light-receiving result obtained by the light receiver and which stores the acquired analysis-related information;
a measuring unit which measures the sample located on the reagent portion of the analysis tool; and
a calculating unit which calculates numerical information in relation to a specified component contained in the sample on the basis of a measurement result of the sample measured by the measuring unit and the analysis-related information stored by the storage unit.
